(19) United States
(12) Patent Application Publication
(10) Pub. No.: US 2021/0386512 A1
(43) Pub. Date: Dec. 16, 2021

(54) Method Tool And Method Of Operating A Machine Tool

(71) Applicant: Ivoclar Vivadent AG, Schaan (LI)

(72) Inventors: Stéphane Follonier, Vilters (CH); Wolfgang Wachter, Schaan (LI)

(21) Appl. No.: 17/281,607

(22) PCT Filed: Sep. 12, 2019
(86) PCT No.: PCT/EP2019/074332

§ 371 Date: Mar. 31, 2021

(30) Foreign Application Priority Data
Oct. 9, 2018 (EP) 18199458.3

(51) Int. Cl. A61C 1/08; G02B 27/01; A61B 34/20; A61B 90/00

(52) U.S. Cl. CPC A61C 1/084; G02B 27/017; A61B 34/20; A61B 2090/365; G02B 2027/0178; A61B 2034/2055; A61B 2034/2065; A61B 90/36

(57) ABSTRACT

The present invention relates to a dental guide system (100) for tooth preparation, comprising an electronic detection system (101) for detecting a three-dimensional actual tooth shape (103-I); a comparing device (105) for ascertaining spatial deviation regions (107), at which the ascertained actual tooth shape (103-I) deviates from a specified desired tooth shape (103-S); and data glasses (109) having a display device (111) for optically displaying the spatial deviation regions (107).

The present invention relates to a dental guide system for tooth preparation and to a method for tooth preparation.

When shaping teeth in the mouth of a patient problems can easily arise because tools, such as e.g. drills, are used incorrectly. Tooth material, once removed, is permanently removed and cannot be added again. Basically, the natural tooth substance should be retained as far as possible.

Therefore, the technical object of the present invention is to simplify and accelerate the manual shaping of teeth in the mouth of a patient.

This object is achieved by the subject matter of the independent claims. Advantageous embodiments are the subject matter of the dependent claims, the description and the figures.

According to a first aspect, the present object is achieved by means of a dental guide system for tooth preparation, comprising an electronic detection system for detecting a three-dimensional actual tooth shape; a comparing device for ascertaining spatial deviation regions, at which the ascertained actual tooth shape deviates from a specified desired tooth shape; and data glasses having a display device for optically displaying the spatial deviation regions. A three-dimensional actual tooth shape can be detected continuously and in real time during shaping. By means of the electronic detection system, a digital data record is obtained which describes the three-dimensional actual tooth shape and which can be processed by the comparing device.

The detection can be performed by means of an optical method or an X-ray method. In general, the electronic detection system can be any detection system which can be used to obtain a digital data record for the three-dimensional tooth shape. The deviation regions are superimposed directly over the visually perceptible tooth. Data glasses having a display device serve to optically display the spatial deviation regions above the visually perceptible tooth.

By optically highlighting deviation regions using the data glasses, a treating dentist can immediately recognise which spatial regions of the tooth are to be removed so that the desired tooth shape is obtained. This provides the technical advantage that inadvertent removal of spatial regions can be prevented and as much natural tooth substance as possible is retained.

The actual tooth shape is e.g. the actual spatial shape of a tooth, a jaw, a drill template replacement, a bridge or a crown. The desired tooth shape is the spatial shape of the tooth, the jaw, the drill template replacement, the bridge or the crown which these are to have after manual processing. The data glasses are an electronic apparatus which are worn as glasses and can be used to optically display further information to a user in addition to natural visual perception. By means of the display device, the data glasses can superimpose additional information on an image which is perceived by the wearer's eye.

The display device comprises e.g. a display screen close to the eyes or a projector for directly projecting on the retina. In addition, the information can be displayed on an external display screen.

In a technically advantageous embodiment of the dental guide system, the data glasses comprise the electronic detection system. By integrating the detection system into the data glasses, e.g. the technical advantage is achieved that the structure of the dental guide system is simplified.

In a further technically advantageous embodiment of the dental guide system, the electronic detection system comprises a stereoscopic detection system having a first and a second camera or a stereoscopic detection system having a plenoptic camera. A plenoptic camera, which is also called a light field camera, detects not only the typical two image dimensions but also the direction of incident light beams. By virtue of the additional dimension, plenoptic pictures contain information relating to the image depth. This provides e.g. the technical advantage that the actual tooth shape can be detected with a small amount of outlay and the detection system can be easily integrated into data glasses. Moreover, the three-dimensional actual tooth shape can also be detected on the basis of a plurality of images by means of a strip projection method or by means of a lenticular camera.

In a further technically advantageous embodiment of the dental guide system, the electronic detection system comprises a 3D camera for detecting a three-dimensional actual tooth shape on the basis of a travel time measurement of light. This provides e.g. the technical advantage that the tooth shape can be ascertained quickly and using a compact camera.

In a further technically advantageous embodiment of the dental guide system, the comparing device is designed to ascertain a restoration parameter on the basis of the detected actual tooth shape. The restoration parameter is e.g. a preparation angle which indicates the angle by which the preparation deviates from the longitudinal axis. However, in general the restoration parameter can be any parameter which plays a role during processing. This provides e.g. the technical advantage that restoration parameters can be monitored and the shaping of the tooth is further simplified.

In a further technically advantageous embodiment of the dental guide system, the guide system is designed to display the restoration parameter on the display device of the data glasses. This provides e.g. the technical advantage that the restoration parameter can be directly taken into consideration during manual processing of the tooth.

In a further technically advantageous embodiment of the dental guide system, the electronic detection system is designed to ascertain the three-dimensional actual tooth shape on the basis of a chronological series of tooth images. This provides e.g. the technical advantage that the tooth shape can be ascertained easily and precisely. Each further tooth image of the series increases the accuracy of the ascertained actual tooth shape. The series of tooth images can be combined e.g. to form a (partial) all-around image of the tooth which is used for further reconstruction of the actual tooth shape. For this purpose, specific computer algorithms can be used.

In a further technically advantageous embodiment of the dental guide system, the electronic detection system is designed to detect the position of a treatment tool. This provides e.g. the technical advantage that the position of the treatment tool can be tracked in real time. The treatment tool can be controlled on the basis of this position. The ascertained position of the treatment tool also makes it possible to deduce the actual shape because no tooth is located at the position of the treatment tool. For example, the three-dimensional actual tooth shape can be calculated using a probing tool as a treatment tool.

In a further technically advantageous embodiment of the dental guide system, the guide system is designed to generate an electronic file which documents a process of a treatment. This provides e.g. the technical advantage that information relating to the treatment can be permanently stored electronically.

In a further technically advantageous embodiment of the dental guide system, the guide system is designed to detect the spatial position of the data glasses. This provides e.g. the technical advantage that the spatial position of the data glasses can be taken into consideration when ascertaining the three-dimensional actual tooth shape. As a result, the accuracy of the ascertained actual tooth shape is increased. By taking an angle into consideration, the accuracy of a projection of the deviation information in the data glasses can be increased.

In a further technically advantageous embodiment of the dental guide system, the detection system is an optical detection system which comprises an autofocus or zoom function for detecting the actual tooth shape. The autofocus function permits automatic focussing by adapting a camera setting to the distance between the camera and tooth and by imaging the tooth in a defined manner. The zoom function allows the image section to be continuously adapted to the tooth. This provides e.g. the technical advantage that the operation and handling of the guide system are simplified.

In a further technically advantageous embodiment of the dental guide system, the guide system comprises a mirror for detecting the three-dimensional actual tooth shape. The mirror provides e.g. the technical advantage that rearward regions of the tooth which are not directly optically accessible can be spatially detected. As a result, a reconstruction of the actual tooth shape can be improved.

According to a second aspect, the present object is achieved by means of a method for tooth preparation, comprising the steps of detecting a three-dimensional actual tooth shape using an electronic detection system; ascertaining spatial deviation regions, at which the ascertained actual tooth shape deviates from a specified desired tooth shape; and displaying the spatial deviation regions on a display device of data glasses. The method provides the same technical advantages as the dental guide system according to the first aspect.

In a technically advantageous embodiment of the method, a restoration parameter is ascertained on the basis of the detected actual tooth shape. The restoration parameter is e.g. a preparation angle of the tooth. This likewise provides e.g. the technical advantage that the shaping of the tooth is further simplified.

In a further technically advantageous embodiment of the method, the restoration parameter is displayed on the display device of the data glasses. This provides e.g. the technical advantage that the restoration parameter can be taken into consideration during manual processing of the tooth.

In a further technically advantageous embodiment of the method, the three-dimensional actual tooth shape is ascertained on the basis of a chronological series of tooth images. This likewise provides e.g. the technical advantage that the tooth shape can be ascertained easily and precisely and each further tooth image of the series increases the accuracy of the ascertained actual tooth shape.

In a further technically advantageous embodiment of the method, the position of a treatment tool is detected. This provides e.g. the technical advantage that the position of the treatment tool can be tracked and a position-based control of the treatment tool can be performed or the actual shape can be deduced from the ascertained position of the treatment tool because no tooth is located at the position of the treatment tool.

In a further technically advantageous embodiment of the method, the spatial position of the data glasses is detected. This likewise provides e.g. the technical advantage that the spatial position of the data glasses can be taken into consideration when ascertaining the three-dimensional actual tooth shape. As a result, the accuracy of the ascertained actual tooth shape is increased. By ascertaining the angle between the data glasses and the tooth position, the spatial deviation regions can be displayed more accurately on the data glasses.

Exemplified embodiments of the invention are illustrated in the drawings and are described in more detail hereinunder.

In the figures:

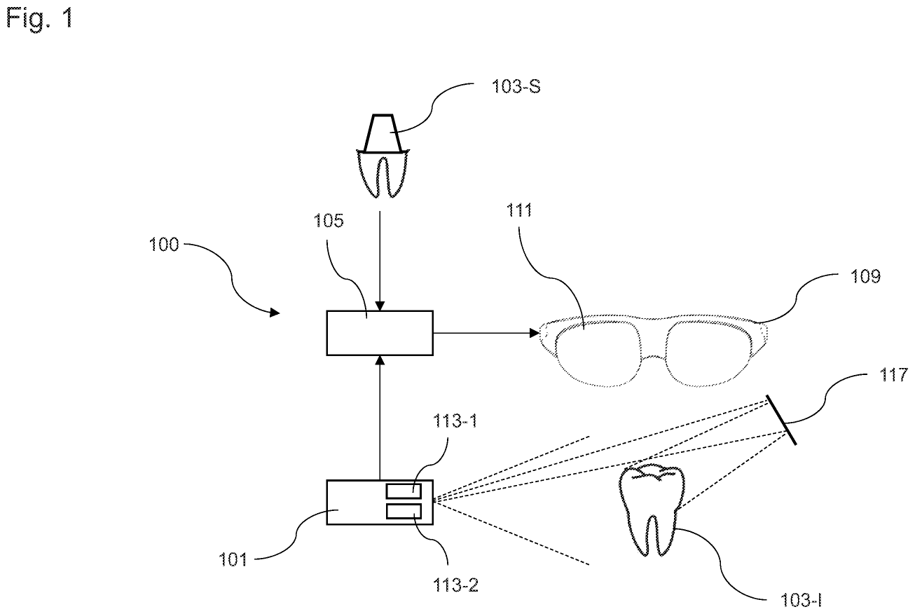

FIG. 1 shows a schematic view of a dental guide system;

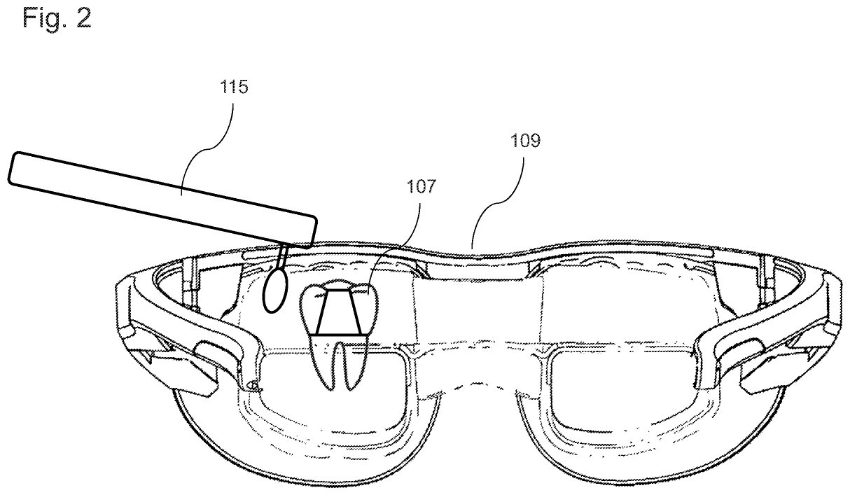

FIG. 2 shows data glasses for viewing a tooth; and

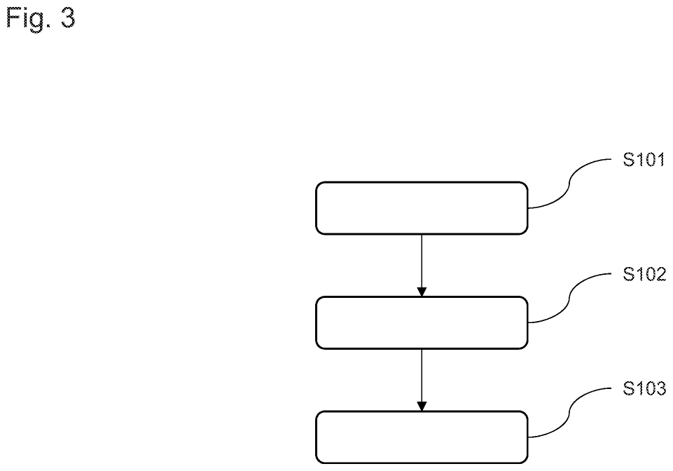

FIG. 3 shows a block diagram of a method for tooth preparation.

FIG. 1 shows a schematic view of a dental guide system 100. The guide system 100 serves to guide a treating person who is performing a tooth preparation in the mouth of a patient. The manual shaping of the tooth is optically assisted by the dental guide system 100. The dental guide system 100 is not only suitable for shaping natural individual teeth but can also be used for shaping artificial teeth or dental aids, such as e.g. bridges, partial prostheses, implants, drill templates or abutments.

For this purpose, the dental guide system 100 comprises an electronic detection system 101, by means of which the three-dimensional actual tooth shape 103-I in the mouth of the patient can be ascertained continuously during shaping. The actual tooth shape 103-I can be ascertained optically. However, for this purpose, it is generally also possible to use any other method, by means of which the spatial actual tooth shape 103-I can be ascertained. The electronic detection system 101 generates e.g. a data record which describes the spatial shape of the tooth.

The detection system 101 comprises e.g. a stereoscopic camera system comprising two electronic cameras 113-1, 113-2. By stereoscopically recording pictures with the two cameras 113-1, 113-2, the spatial actual tooth shape 103-I can be easily ascertained and reconstructed. From the respective images of the cameras 113-1, 113-2 at different parallax angles it is possible to calculate the actual tooth shape 103-I using a computer algorithm.

The detection system 101 can also comprise a camera which ascertains individual distances using a travel time method of light (TOF (time of flight) camera). For this purpose, the tooth is illuminated by means of a light pulse and the time required by the light to travel to the object and back again is measured for each image point. This time is directly proportional to the distance. The camera provides the distance of the tooth imaged thereon for each image point.

The detection system 101 can also ascertain the actual tooth shape 103-I on a series of tooth images which have been obtained from different viewing angles. For this purpose, a calculating method can be used which reconstructs the actual tooth shape 103-I from the individual tooth images. In addition, information relating to the spatial position of the detection system 101 can be used for this purpose.

However, the detection system 101 can also be based upon a computer tomography method, X-ray method or a magnetic resonance method in order to ascertain the actual tooth shape 103-I. This provides the advantage that three-dimensional shapes which are optically not accessible, such as e.g. tooth shapes including lesions, can also be determined. Therefore, the internal situation and the internal volume of the tooth can be detected by these methods. For example, if the tooth has a hole then a corresponding actual tooth shape can be obtained which reproduces not only the external visually perceptible surface. The spatial shape of the hole can be displayed via the display device 111 so that even in this case the generation of the desired tooth shape can be assisted. In the case of minimally invasive methods, the position of the drill hole can thus be determined.

The ascertained actual tooth shape 103-I is supplied to an electronic comparing device 105 as a data record. The comparing device 105 ascertains the spatial deviation regions 107, at which the ascertained actual tooth shape 103-I deviates from a specified desired tooth shape 103-S.

The desired tooth shape 103-S is selected e.g. beforehand in an intermediate step via a user interface or is suggested automatically by reference to clinical cases, e.g. from a database or by algorithms (machine learning) or is specifically configured, e.g. using a CAD method and is likewise supplied to the electronic comparing device 105 as a data record. A database can store a number of data records for possible desired tooth shapes. This host of desired tooth shapes can be selected either automatically or manually. Moreover, it is possible to change specified desired tooth shapes via a user interface.

In order to ascertain the deviation, e.g. the data record which describes the spatial actual tooth shape 103-I is compared to the other data record which describes the desired tooth shape 103-S. If e.g. the actual tooth shape and the desired tooth shape 103-I, 103-S do not correspond within the scope of a specified tolerance value, the corresponding region is detected as a deviation region 107.

The electronic comparing device 105 is formed e.g. by a software module which is executed on a computer device having a processor and an electronic memory for storing the software module and the data records.

The spatial deviation regions 107 ascertained in this way are communicated to the data glasses 109, which assists the tooth preparation. The data glasses 109 optically accentuate the deviation regions 107.

The data glasses 109 (also called augmented-reality-glasses or smart glasses) are a wearable apparatus which is able to virtually project information in front of the eyes of the wearer of the glasses while said wearer is still able to see the surrounding area. As a result, information can be displayed and added in the field of vision of the wearer. For this purpose, the data glasses 109 comprise a display device 111 which can be formed from a display screen in close proximity to the eyes or a projector for directly projecting on the retina.

The display device 111 serves to display those regions, at which the ascertained actual tooth shape 103-I deviates from the specified desired tooth shape 103-S. In this way, a wearer of the data glasses 109 can recognise immediately on the visually perceived tooth which spatial regions of the tooth must be removed in order to obtain the desired tooth shape 103-S. The wearer of the data glasses 109 uses a dental tool, such as e.g. a drill or cutter, in order to remove the accentuated spatial regions of the tool.

The data glasses 109 can additionally also comprise sensors for detecting movement of the head or for recognising the spatial position of the data glasses 109, such as e.g. a gyro sensor. Therefore, the display of the calculated deviation regions 107 can be adapted more precisely to the movements of the wearer. The spatial position of the data glasses 109 can be affected e.g. on the basis of specified optical reference points or markers which are disposed in the area surrounding the data glasses 109, such as e.g. by means of trilateration or triangulation.

The detection system 101 can be integrated into the data glasses 109. This provides the technical advantage that the actual tooth shape 103-I can be easily performed whilst wearing the data glasses 109. In general, the detection system 101 can also be provided as a separate apparatus, with which the actual tooth shape 103-I is ascertained during shaping.

FIG. 2 shows the data glasses 109 for viewing the tooth and for displaying the deviation regions 107. The deviation regions 107 which are to be removed are superimposed directly over the visually perceptible tooth. As a result, the wearer of the data glasses 109 can recognise in real time during tooth preparation the points at which the actual tooth shape 103-I deviates from the desired tooth shape 103-S.

The desired tooth shape 103-S can be selected manually on the basis of a library which contains a host of desired tooth shapes 103-S. Possible desired tooth shapes 103-S can be displayed to the user on the data glasses 109 by means of the display device 111.

However, it is also possible for the comparing device 105 to select the desired tooth shape 103-S on the basis of the previously detected actual tooth shape 103-I. In this case, from a host of specified desired tooth shapes 103-S it is possible to select the one desired tooth shape, with which the most tooth substance is retained in comparison with the actual tooth shape 103-I.

Moreover, the comparing device 105 can be designed to automatically ascertain a restoration parameter on the basis of the detected actual tooth shape 103-I. This can be e.g. a preparation angle which indicates the angle by which the present actual tooth shape 103-I deviates from a crown longitudinal axis, i.e. e.g. a half cone angle. For this purpose, the preparation angle of the actual tooth shape 103-I is ascertained on the basis of the continuously detected actual tooth shape.

The wearer of the data glasses 109 receives the current preparation angle displayed thereon so that said wearer can process the tooth until the correct preparation angle, i.e. the angle of the prepared stump, is achieved.

However, in general the restoration parameter can also describe other properties, such as e.g. other geometric parameters relating to a tooth stump, an implant, an abutment, undercuts, jawbone situations or insertion angle, or parameters relating to the aesthetic situation, such as e.g. a colour, a point on the tooth, the gingiva, papilla, palatal ridge or gums. In addition, the electronic detection system 101 can detect the position of a treatment tool 115 or a mirror 117.

Furthermore, the guide system 100 can be designed to continuously detect the shaping of the tooth by means of the detection system 101 and to generate an electronic file which completely documents a sequence of a treatment, such as a video file or CAD file. In this case, the shaping of the tooth can also be retrospectively tracked and appraised on the basis of the electronic file.

Moreover, the detection system 101 can be designed to optically detect not only the actual tooth shape but also the tooth colour of the tooth, e.g. by obtaining digital colour values of the tooth colour during a recording. These colour values can be stored in the respective data record in addition to the detected actual tooth shape.

Moreover, the guide system can have a mirror 117 to assist in the detection of the three-dimensional actual tooth shape. By virtue of the mirror 117, the detection of the actual tooth shape is accelerated, in that rearward regions of the tooth which are not directly optically accessible, such as e.g. an inner side of the tooth, can be spatially detected. Additional tooth images can be ascertained easily and rapidly by virtue of the mirror 117, said images being able to be combined e.g. to form a (partial) all-around image of the tooth which is used for further reconstruction of the actual tooth shape. In general, suitable computer algorithms can be used in order to ascertain the tooth shape.

The mirror 117 can be connected e.g. in a fixed manner to the optical detection device 111 so that optical detection can be performed simultaneously both on the front side and rear side of the tooth.

This can be e.g. a mirror having an integrated camera/video system.

However, in general the mirror can also be a hand-held mirror 117 which can be used to obtain tooth images of the rear side of the tooth. These tooth images can likewise be used for reconstruction of the actual tooth shape. Moreover, the detection device 101 can detect the position of the mirror 117 so that this position can be taken into consideration when the actual tooth shape is being ascertained, such as e.g. by means of a ray-tracing method.

FIG. 3 shows a block diagram of a method for tooth preparation. The method comprises the step S101 of detecting the three-dimensional actual tooth shape 103-I using the electronic detection system 101, the step S102 of ascertaining spatial deviation regions 107, at which the ascertained actual tooth shape 103-I deviates from the specified desired tooth shape 103-S; and the step S103 of displaying the spatial deviation regions 107 on the display device 111 of the data glasses 109.

All features explained and illustrated in conjunction with individual embodiments of the invention can be provided in different combinations in the subject matter in accordance with the invention in order to achieve the advantageous effects thereof at the same time.

All the method steps can be implemented by devices which are suitable for carrying out the respective method step. All functions which are carried out by features relating to an apparatus can be a method step of a method.

The scope of protection of the present invention is set by the claims and is not limited by the features explained in the description or shown in the figures.

### LIST OF REFERENCE SIGNS

100 dental guide system
101 detection system
103-I actual tooth shape
103-S desired tooth shape
105 comparing device
107 deviation region
109 data glasses
111 display device
113-1 camera
113-2 camera
115 treatment tool
117 mirror

1. A dental guide system (100) for tooth preparation, comprising:
an electronic detection system (101) for detecting a three-dimensional actual tooth shape (103-I);
a comparing device (105) for ascertaining spatial deviation regions (107), at which the ascertained actual tooth shape (103-I) deviates from a specified desired tooth shape (103-S);
data glasses (109) having a display device (111) for optically displaying the spatial deviation regions (107).

2. The dental guide system (100) as claimed in claim 1,
wherein the data glasses (109) comprise the electronic detection system (101).

3. The dental guide system (100) as claimed in claim 1,
wherein the electronic detection system (101) comprises a stereoscopic detection system having a first and a second camera (113-1, 113-2) or a stereoscopic detection system having a plenoptic camera.

4. The dental guide system (100) as claimed in claim 1,
wherein the electronic detection system (101) comprises a 3D camera for detecting a three-dimensional actual tooth shape (103-I) on the basis of a travel time measurement of light.

5. The dental guide system (100) as claimed in claim 1,
wherein the comparing device (105) is designed to ascertain a restoration parameter on the basis of the detected actual tooth shape (103-I).

6. The dental guide system (100) as claimed in claim 5,
wherein the guide system (100) is designed to display the restoration parameter on the display device (111) of the data glasses (109).

7. The dental guide system (100) as claimed in claim 1,
wherein the electronic detection system (101) is designed to ascertain the three-dimensional actual tooth shape (103-I) on the basis of a chronological series of tooth images.

8. The dental guide system (100) as claimed in claim 1,
wherein the electronic detection system (101) is designed to detect the position of a treatment tool (115).

9. The dental guide system (100) as claimed in claim 1,
wherein the guide system (100) is designed to generate an electronic file which documents a process of a treatment.

10. The dental guide system (100) as claimed in claim 1,
wherein the guide system (100) is designed to detect the spatial position of the data glasses (109).

11. The dental guide system (100) as claimed in claim 1,
wherein the detection system (111) is an optical detection system which comprises an autofocus or zoom function for detecting the actual tooth shape.

12. The dental guide system (100) as claimed in claim 1,
wherein the guide system (100) comprises a mirror (117) for detecting the three-dimensional actual tooth shape (103-I).

13. A method for tooth preparation, comprising the steps of:
detecting (S101) a three-dimensional actual tooth shape (103-1) using an electronic detection system (101);
ascertaining (S102) spatial deviation regions (107), at which the ascertained actual tooth shape (103-I) deviates from a specified desired tooth shape (103-S); and
displaying (S103) the spatial deviation regions (107) on a display device (111) of data glasses (109).

14. The method as claimed in claim 13,
wherein a restoration parameter is ascertained on the basis of the detected actual tooth shape (103-I).

15. The method as claimed in claim 14,
wherein the restoration parameter is displayed on the display device (111) of the data glasses (109).

16. The method as claimed in claim 13,
wherein the three-dimensional actual tooth shape (103-I) is ascertained on the basis of a chronological series of tooth images.

17. The method as claimed in claim 13,
wherein a position of a treatment tool (115) is detected.

18. The method as claimed in claim 13,
wherein the spatial position of the data glasses (109) is detected.
